(19) United States
(12) Patent Application Publication
(10) Pub. No.: US 2016/0074438 A1
(43) Pub. Date: Mar. 17, 2016

(54) PHARMACEUTICAL COMPOSITIONS FOR TREATING DEGENERATIVE NEUROLOGICAL DISEASE WITH MITOCELLS

(71) Applicant: Taiwan Mitochondrion Applied Technology Co., Ltd, Zhubei City (TW)

(72) Inventors: Shinn-Zong LIN, Taichung (TW); Horng-Jyh HARN, Taichung (TW); Shih-Ping LIU, Taichung (TW); Han-Chung CHENG, Zhubei City (TW); Chi-Tang TU, Zhubei City (TW)

(21) Appl. No.: 14/851,402

(22) Filed: Sep. 11, 2015

Related U.S. Application Data
(60) Provisional application No. 62/049,090, filed on Sep. 11, 2014.

(51) Int. Cl. A61K 35/35; C12N 5/0775

(52) U.S. Cl. CPC A61K 35/35; C12N 5/0667; C12N 2501/999

(57) ABSTRACT

A MitoCells treated with angelica extract is provided. The pharmaceutical composition comprise MitoCells and can significantly achieve the goal for treating degenerative neurological disease.

### CROSS-REFERENCE TO RELATED APPLICATIONS

This Non-provisional application claims priority under 35 U.S.C. §119(a) on Patent Application No(s). [62/049,030] filed in United States America [Sep. 11, 2014], the entire contents of which are hereby incorporated by reference.

### FIELD OF THE INVENTION

The present invention relates to a pharmaceutical composition, and particularly relates to a pharmaceutical composition for treating degenerative neurological disease and improving the differentiation of cells from stem cells into neurons.

### BACKGROUND OF THE INVENTION

Because of advances in economic development and health care, the average age of the population is increasing by the numbers of older people. Population aging has occurred as a global trend. According to the report of United Nations, the world population in 2012 is about 7.08 billion. And the population over 65 years in worldwide is 7.9% of the total population in 2012. This is an aging society defined by World Health Organization (WHO). Refer to the world population is ageing, and the patient numbers of neurodegenerative diseases are rapidly increased, and more than 400 million worldwide people suffer degenerative nerve diseases. However, the neurodegenerative disease not only occurs in the elderly, and about 50% people suffer the neurodegenerative disease before age 60. The other 50% people suffer the neurodegenerative disease after age 60. Neurodegenerative disease is a disorder condition of progressive degeneration in brain or spinal neurons, which results from the destruction or loss of the synapse and myelin sheath. The disorder leads to function disturbance, walking difficulties, and death.

Parkinson's disease is more common in older people, with the most cases are occurring after the age of 50 to 79. It is characterized by the death of dopaminergic neurons in the substantia nigra. Substantia nigra has about 200,000 dopaminergic neurons in of normal human tissues. Dopaminergic neurons secrete the neurotransmitter dopamine and play important roles in neurological functions including coordinated motion control. If the degeneration is not serious, it will not cause uncoordinated movements. However, when more than 50% neurons in human are degenerated, mild symptoms may occur in the patients including shaking, rigidity, slowness of movement and difficulty with walking and gait. Later, thinking and behavioral problems may arise, with dementia commonly occurring in the advanced stages of the disease. Finally, the patients may die from respiratory tract infection, urinary tract infection, and bedsore.

Currently, the treatment of Parkinson's disease in the early stage is typically with the medications L-DOPA to increase dopamine concentrations to maintain normal dopamine concentration in blood. For early Parkinson's disease, using L-DOPA medicine can make good treatment. But the disease progresses and dopaminergic neurons are continuing lost, these drugs eventually need to take more and more, but the symptoms get more serious. Finally, the drugs become ineffective. Most people who use these medicines for many years may cause the adverse side effects including hallucinations, nausea, gastrointestinal upset, and involuntary dancing movements. Since the drugs are unable to control the symptoms in the late stage of treatment, surgery will be used to improve the quality of the life. Surgery for Parkinson's disease can be divided into three main groups: (1) Cautery incision. Target areas for lesions include the globus pallidus, thalamus, and hypothalamus nucleus. These areas are heated with 80° C. about 80 seconds to inactive the function of neuron cells; (2) Implantation of electrodes, which is similar to (1). Electrodes are inserted into the brain to reduce physical shaking; and (3) stem cell therapy. Stem cells are used to supply a source of dopaminergic neurons to replace the function of those cells lost during the neurodegenerative process and improve the symptoms of Parkinson's disease.

Although, stem cell treatment for Parkinson's disease is published, and it can improve the symptoms of Parkinson's disease. However, the survival rate of stem cells and differentiation of the stem cells into dopaminergic neurons in patients after injection are low (Cave et al, 2014). Thus, the stem cell treatment still cannot treat or cure the neurodegenerative disease.

### SUMMARY OF THE INVENTION

In view of the above-mentioned problem, the present invention provides a pharmaceutical composition comprising a MitoCell. The MitoCell is an adipose stem cell that is pre-treated with an angelica extract to induce the differentiation of the adipose stem cell into neurons in vitro. After the MitoCell is administered to a subject, there is a high-ratio differentiation of stem cells into neurons. The pharmaceutical composition can increase the differentiation from stem cells into neurons and suppress their immune response to achieve the treatment of Parkinson's disease.

The present invention provides a medium for a MitoCell, comprising an angelica extract.

In one embodiment, the angelica extract comprises butylidenephthalide.

The present invention also provides a method for preparing a MitoCell, comprising pre-treating a stem cell with an angelica extract.

The present invention further provides a MitoCell, which is derived from a stem cell treated with the angelica extract.

In one embodiment, the MitoCell is a stem cell.

In one embodiment, the MitoCell is an adipose stem cell.

In one embodiment, according to JC-1 fluorescence dye staining, the ratio of red/green fluorescence of the mitochondria membrane potential of MitoCells is 6.5 to 2.7.

The present invention further provides a pharmaceutical composition for increasing neurons, comprising 50 to 90% MitoCells.

The present invention further provides a method for treating degenerative neurological disease, comprising administering MitoCells into brain of a subject.

### BRIEF DESCRIPTION OF THE DRAWINGS

FIG. 1A-1B illustrate the survival rate of the stem cells in the angelica extract with various concentration.

FIG. 2 illustrates that the secretion of the neurotrophins Nurr1and BDNF is increased in adipose stem cells treated with the angelica extract with various concentration. The results indicate that the adipose stem cells are induced to differentiate into neurons. The increase of SDF1 indicates improvement of stem cell homing, and the decrease of IL-8 indicates the suppression of inflammation.

FIG. 3A illustrates the ratio of red/green fluorescence is decreased in mitochondria of MitoCells. The membrane potential of mitochondria of MitoCells is different from that of normal cells. FIG. 3B illustrates the MitoCells still have the essential stem cell characteristics (CD44/CD 105).

FIGS. 4A-4B illustrate the results of Beam walking test. The results show that after the mice were administrated with adipose stem cells (Group 3) or MitoCells (Group 4), the activities on balance control of the mice was significantly improved, and the activities on balance control in Group 4 was better than Group 3.

FIG. 5 illustrates the results of rotarod test. The results indicated that after the mice were administrated with adipose stem cells (Group 3) or MitoCells (Group 4), the coordination and balance of mice were recovered, and the recovery in Group 4 was better than Group 3.

FIGS. 6A-6C illustrate the results of locomotor activity box test. The results indicated that after the mice were administrated with adipose stem cells (Group 3) or MitoCells (Group 4), the behavior ability of mice was recovered, and the recovery in Group 4 was better than Group 3.

FIG. 7 illustrates the results of brain sections stained by H&E (Hematoxylin and Eosin). The results indicated that adipose stem cells and MitoCells did not have toxicity to neurons and would not enhance the inflammation in brain after injection.

### DETAILED DESCRIPTION OF THE INVENTION

The present disclosure is directed to novel fusion proteins comprising a bioactive molecule and portions of an immunoglobulin molecule. Various aspects of the present disclosure relate to fusion proteins, compositions thereof, and methods for making and using the disclosed fusion proteins. The disclosed fusion proteins are useful for extending the serum half-life of bioactive molecules in an organism.

The following is a detailed description provided to aid those skilled in the art in practicing the present invention. Those of ordinary skill in the art would understand that modifications or variations of the embodiments expressly described herein, which do not depart from the spirit or scope of the information contained herein, are encompassed by the present disclosure. The terminology used in the description is for describing particular embodiments only and is not intended to be limiting of the invention. The section headings used below are for organizational purposes only and are not to be construed as limiting the subject matter described.

Angelica can be dried by freeze drying, spray drying, evaporation, or heat drying, etc. In the present invention, the term “angelica” as used herein refers to a taproot, lateral root, or fibers of Angelica sinensis. The angelica can be extracted using an agent to obtain an angelica extract. For example, a supercritical fluid extraction, water extraction, or organic solvent extraction method can be used. Preferably, the angelica extract of the present invention comprises butylidenephthalide.

The term “stem cell” as used herein, refers to a cell in an undifferentiated or partially differentiated state that has the property of self-renewal and has the developmental potential to differentiate into multiple cell types, without a specific implied meaning regarding developmental potential. The stem cell includes embryonic and adult stem cells. Natural somatic stem cells have been isolated from a wide variety of adult tissues including blood, bone marrow, brain, olfactory epithelium, skin, pancreas, skeletal muscle, and cardiac muscle. The stem cells of the invention include, but are not limited to, adipose stem cells, neural stem cells, neural crest stem cells, mesenchymal stem cells, hematopoietic stem cells, pancreatic stem cells, hematopoietic stem cells, skin stem cells, embryonic stem cells, endothelial stem cells, liver stem cells, intestinal epithelial stem cells and germ stem cells, preferably adipose stem cells.

The present invention provides a MitoCell. The MitoCells of the present invention is obtained by treating a stem cell with a medium containing the angelica extract and/or butylidenephthalid for at least 1 hours, preferably more than 2, 3, 4, 5, 6, 7, 8, 9, 10, 15, 20, or 24 hours, more preferably, more than 1, 2, 3, 4, 5, 6, 7, 8, 9, or 10 days.

It shall be noted that the MitoCells still remain the properties of stem cells after treated with angelica extract. Additionally, the MitoCells can be differentiated into neurons in mice.

When the stem cells are treated with angelica extract, the mitochondria of the cells are activated and the treated stem cells still remain the characteristic of stem cells. For example, the surface markers CD44+/CD 105+ can be detected in all of the treated stem cells both before and after treatment.

The present invention provides a method for preparing a MitoCell, comprising culturing a stem cell in a medium, wherein the medium comprises an angelica extract.

The present invention also provides a MitoCell for preparing a pharmaceutical composition for treating degenerative neurological disease, characterized in that the MitoCells are injected into a brain of a subject.

The present invention further provides a pharmaceutical composition. The pharmaceutical composition of the present invention comprises MitoCells, wherein the MitoCell is present in an effective amount from about 50% to 90% of the formulation, particularly, 80% to 90% of the formulation.

The pharmaceutical composition can effectively improve the quantity and quality of neurons in brain to improve the balance and coordination abilities of the subjects. The subject of the present invention includes a human or non-human animals (e.g., mouse, dog, cat, sheep, cattle, horse, or monkey, etc), preferably, human.

It is important that the MitoCells not only effectively increase the amount of dopaminergic neurons, but also decrease the subject's immune response caused by MitoCells. The MitoCells are better than untreated stem cells.

The pharmaceutical composition of the present invention can be administered alone or combined with other methods or drugs of treatment of degenerative neurological disease.

As mentioned above, MitoCells of the present invention can increase the amount of dopaminergic neurons in brain, particularly in substantia nigra, to treat degenerative neurological disease, such as Parkinson's disease or Alzheimer's disease. Additionally, the risk of immune rejection of MitoCells is lower than adipose stem cells.

Additional specific embodiments of the present invention include, but are not limited to the following:

### EXAMPLE 1

### Culture and Pre-Treatment of MitoCell

MitoCells were prepared by culturing the stem cells in an adipose stem cell medium. The adipose stem cell medium included Keratinocyte-SFM (1X) liquid (Gibco), bovine pituitary extract (Gibco), EGF (Gibco), N-acetyl-L-cysteine (Sigma), L-ascorbic acid phosphate magnesium salt hydrate (Sigma), 10% bovine Serum (HyClone), and 0, 5, 10, 20, 40, 80, 160, and 320 μg/μl angelica extract (butylidenephthalide), respectively. The following term “MitoCell” is defined as the adipose stem cell treated with angelica extract.

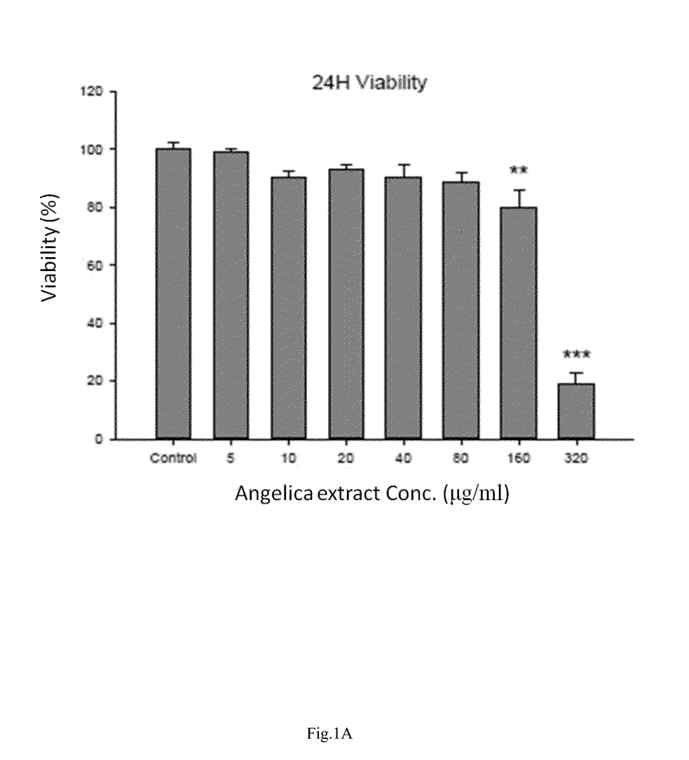

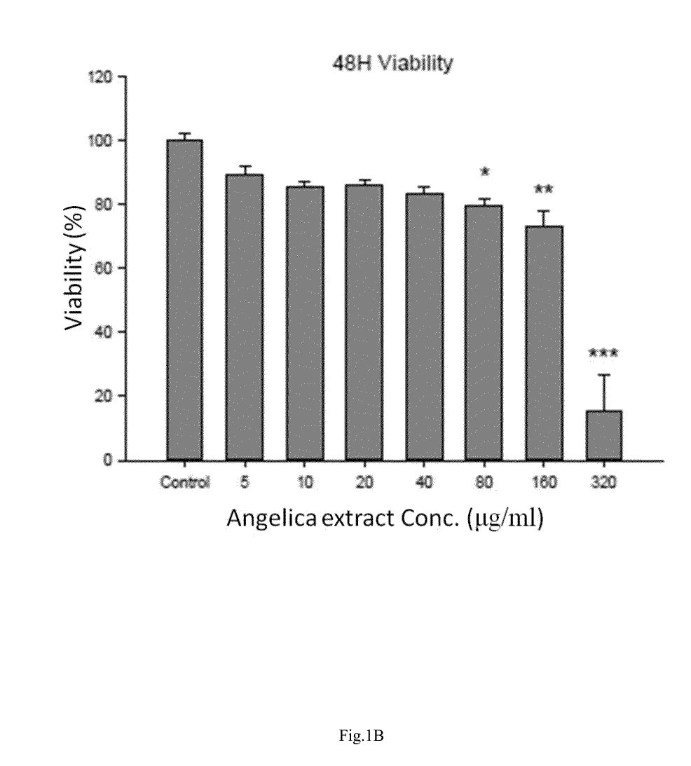

Referring to FIG. 1, after 24 hours of culture, the survival rate of the MitoCells was decreased when the concentration of the angelica extract was more than 160 μg/mL. After 48 hours of culture, the survival rate of the MitoCells was decreased when the concentration of the angelica extract was more than 80 μg/mL.

Additionally, the adipose stem cells were cultured in 0, 0.3125, 0.625, 1.25, 2.5, 5, and 20 μg/mL angelica extract, respectively. The expression of Nurr1, BDNF, SDF1, and IL-8 genes in MitoCells was analyzed to determine the optimal dose in the treatment.

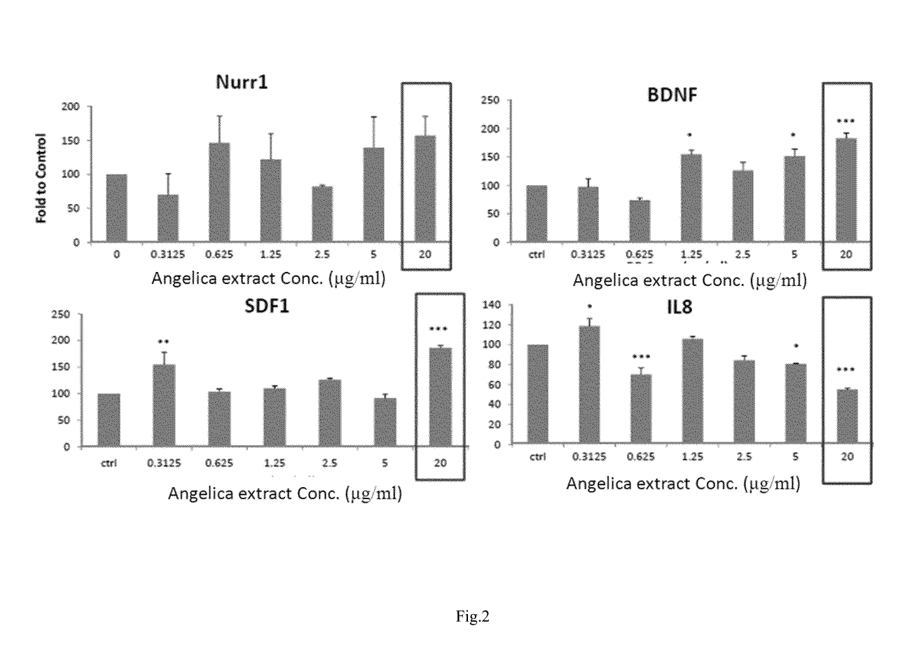

As shown in FIG. 2, the expression of Nurr1, BDNF, and SDF1 genes was increased, but the expression of IL-8 gene was suppressed at high concentration (20 μg/mL) of the angelica extract.

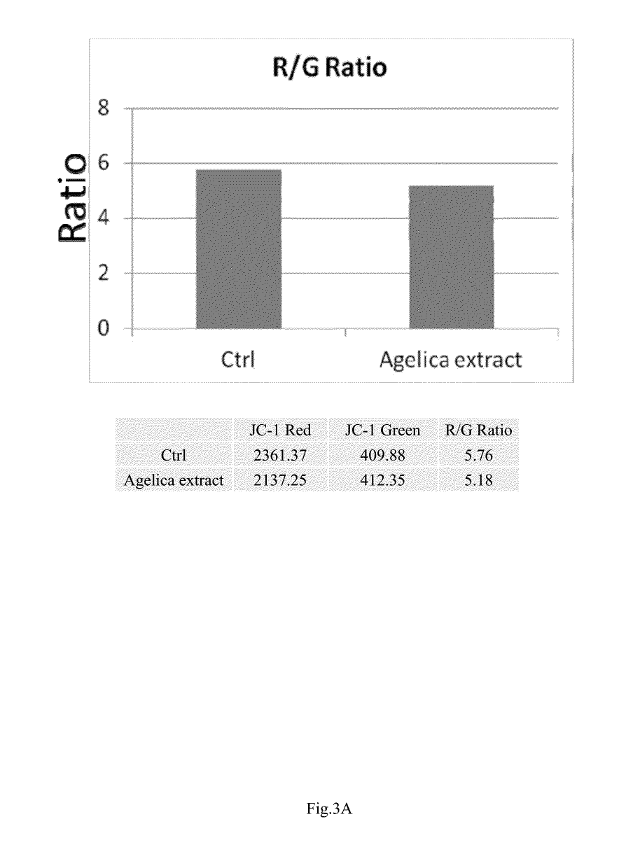

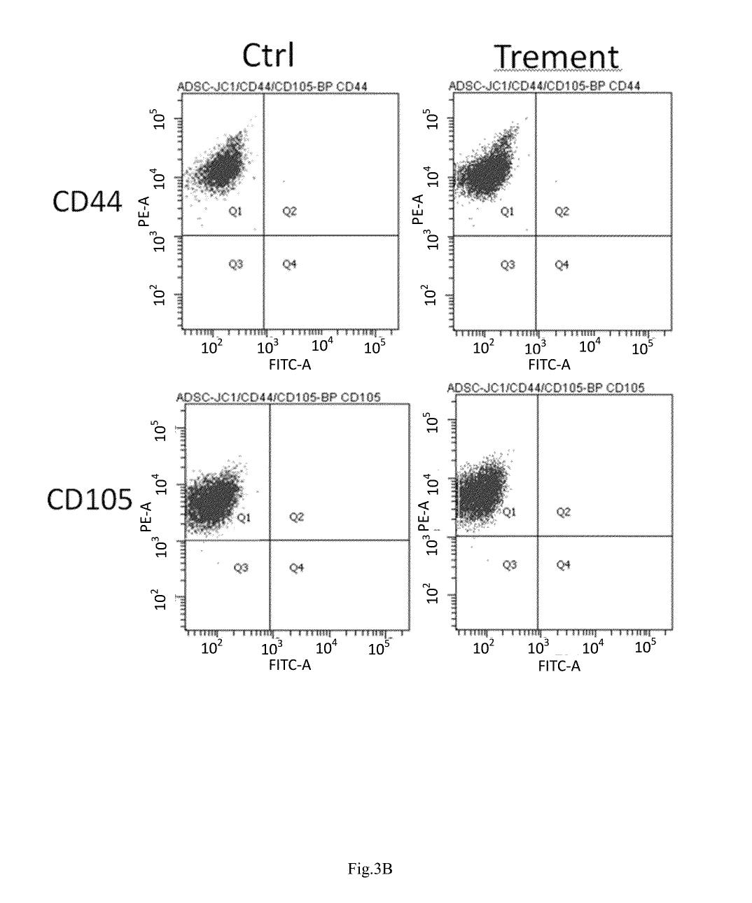

Referring to FIG. 3, FIG. 3A shows a change of JC-1 staining red/green fluorescence ratio of mitochondria in MitoCells. The changes of mitochondria membrane potential of MitoCells was significant. FIG. 3B shows the flow cytometry analysis of the MitoCells. The expression of the cell markers CD44+/CD105+ indicated that MitoCells still have the essential stem cell characteristics. 20 μg/mL of angelica extract was selected for the following tests.

### EXAMPLE 2

### Establishment of Induced Parkinson's Disease Mouse Model

C57BL/6 male Mice (eight weeks old), weighing 25 g, were purchased and used in this Example. After mice were divided into four groups, a few days of adaptation was provided to avoid stress and anxiety to affect the experimental process and the results of analysis. One day before the experiment, neurobehavioral observations and analysis were carried out first. Ten minutes before the surgery, 4% cholra hydrate was administered to mice at a dosage of 1 mL/g/Kg bodyweight. 0.25 mL of 4% chloral hydrate was administered to mice with a body weight of 25 g. Further, mice were anesthetized with isoflurane to prevent the mice waking up during the surgery.

1-methyl-4-phenyl-1,2,3,6-tetrahydropyridine (MPTP) dissolved in saline was used to induce Parkinson's disease in mice. The mice were administered with MPTP four times daily intraperitoneal (I.P.) injection with a 2-hours interval between injections at a dosage of 20 mg/kg. 1×10⁶ cells were injected to mice in experimental groups as shown in Table 1.

(1) Group 1 (Control Group): No MPTP Injection

(2) Group 2 (Negative Control Group): MPTP Injection to Induce Parkinson's Disease+Saline

(3) Group 3 (Experimental Group): MPTP Injection to Induce Parkinson's Disease+1×10⁶ Adipose Stem Cells

(4) Group 4 (Experimental Group): MPTP Injection to Induce Parkinson's Disease+1×10⁶ MitoCells

### EXAMPLE 3

### Neurobehavioral Analysis After/Before Surgery

3.1 Beam Walking Analysis

Beam walking test was used to analyze the balance ability of mice. Mice were placed at the extremity of a 80 cm-long wooden narrow beam and record the time spent in walking and the number of foot slips to analyze the balance and coordination of mice. Test time was 60 seconds. If the mice could not traverse the entire beam successfully within 60 seconds, the spent time was recorded as 60 seconds.

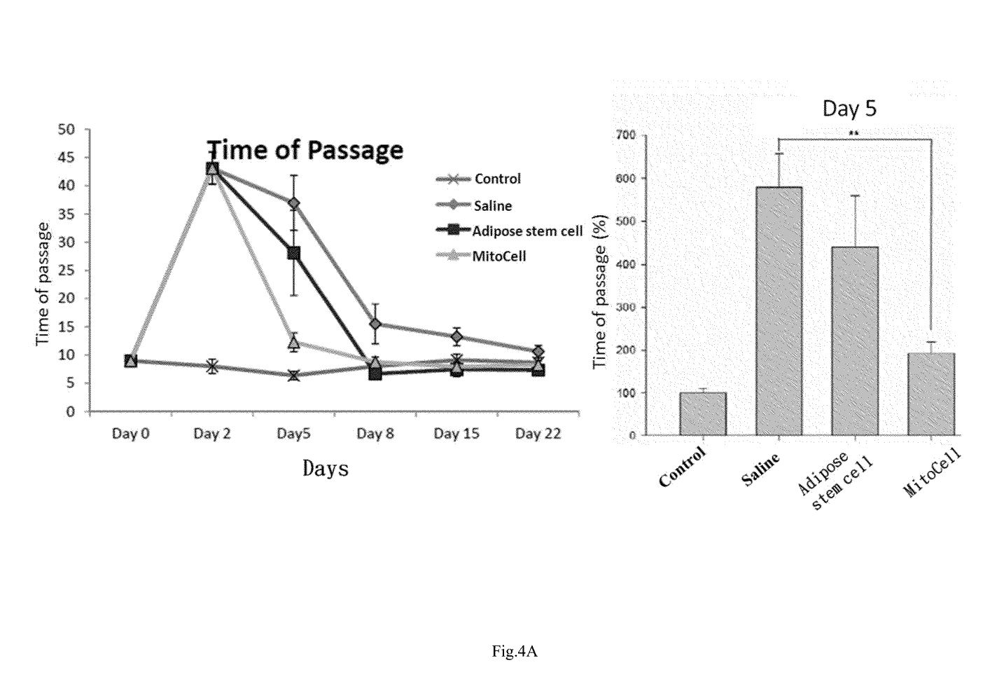

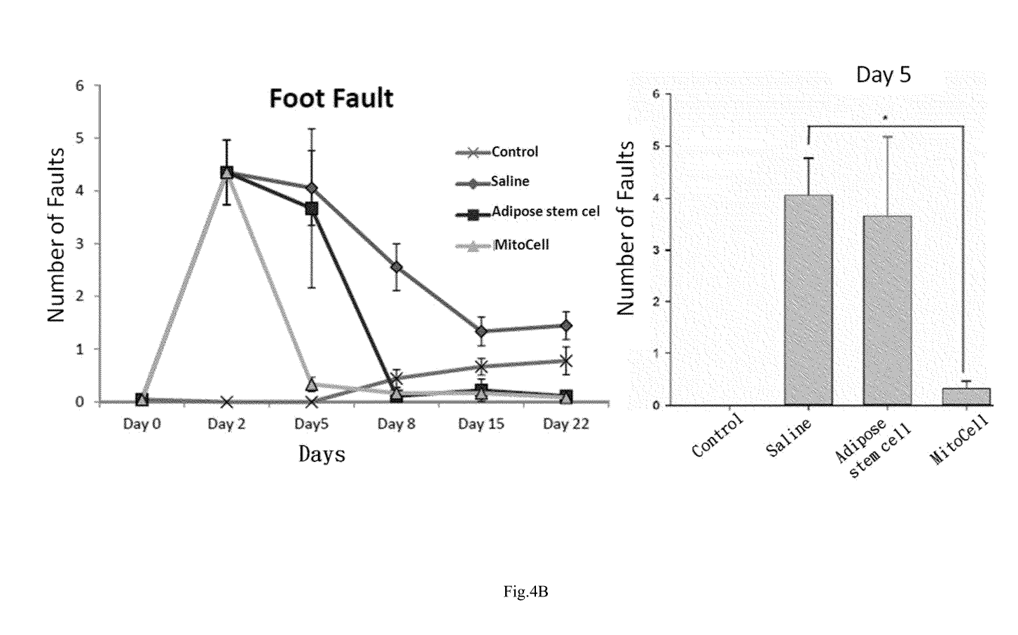

Referring to FIG. 4A, MPTP-induced Parkinson's disease model mice (Group 2) could not complete the beam walking test. After rejection of adipose stem cells or MitoCells (Groups 3 and 4), the balance abilities of mice were significantly improved.

Referring to FIG. 4B, the number of foot slips was increased in mice after administration of MPTP (Group 2). After rejection of adipose stem cells or MitoCells (Groups 3 and 4), the number of foot slips was decreased.

The results indicated that MitoCells (Group 3) had a better treatment effect in mice compared to adipose stem cells (Group 4).

3.2 Rotarod Analysis

Rotarod analysis was used to determine the balance and coordination abilities of mice. One week before the experiment, the mice were trained to perform on the rotarod at 3 minutes. After surgery, the recovery of balance in mice was analyzed by rotarod analysis at 5 rpm.

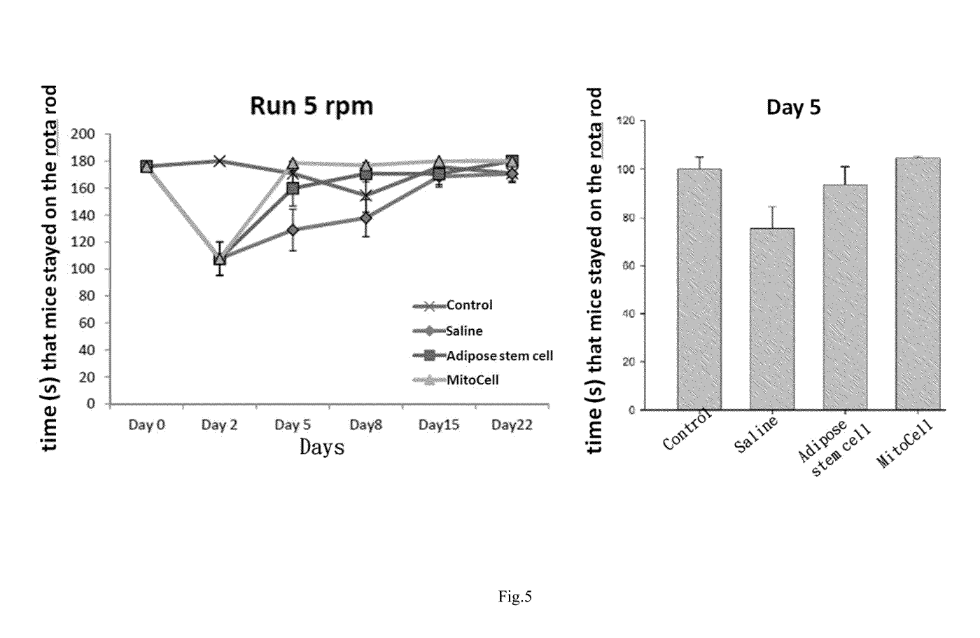

Referring to FIGS. 5A and 5B, the balance and coordination abilities in mice were significantly decreased after administration of MPTP (Group 2). However, after rejection of adipose stem cells or MitoCells (Groups 3 and 4), the balance and coordination abilities in mice were recovered, preferably rejection of MitoCells (Group 4).

3.3 Locomotor Activity Box

Before the monitor, Mice were placed in the chamber for 10 to 20 minutes to adapt the environment. The locomotor activity box was connected to a computer to monitor and record the mouse locomotor activities including running (horizontal locomotion), head rising/climbing, and total distance traveled for 30 minutes. The data were collected for statistical analysis

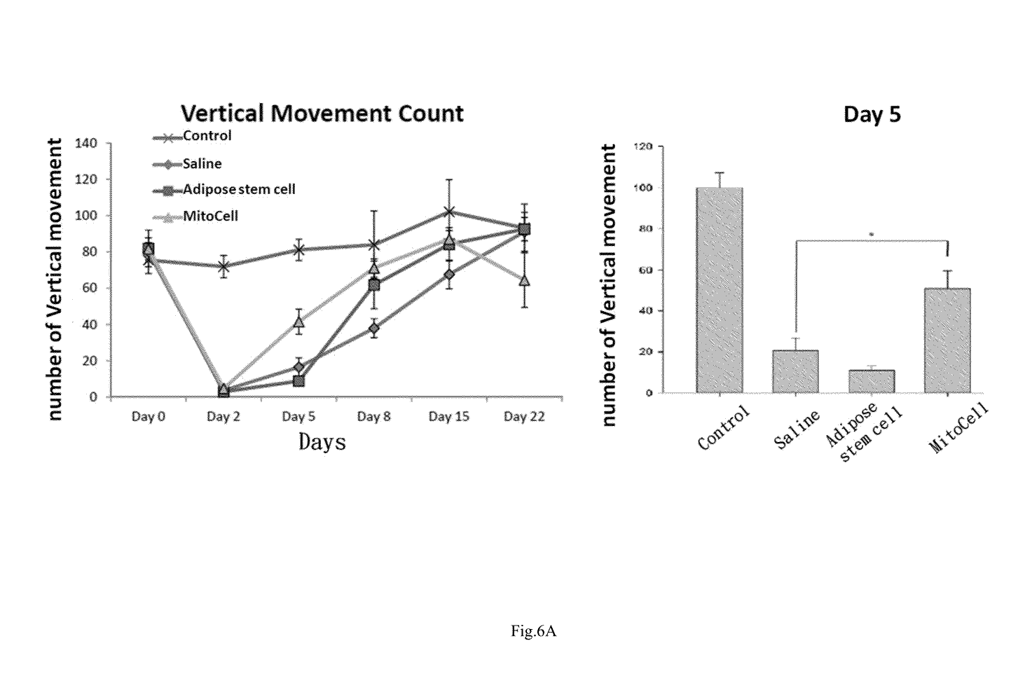

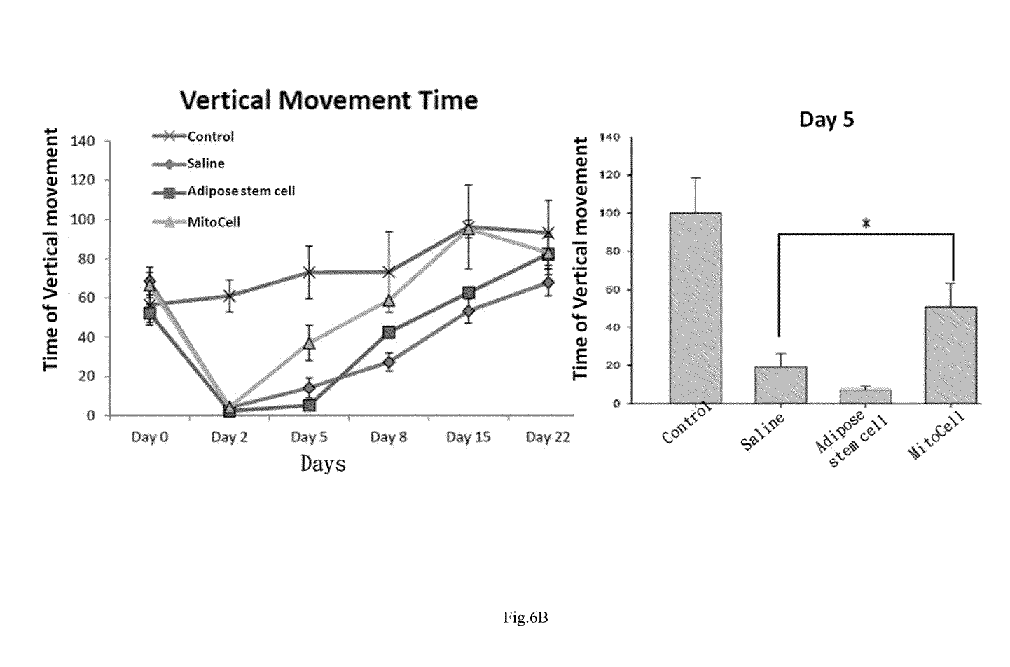

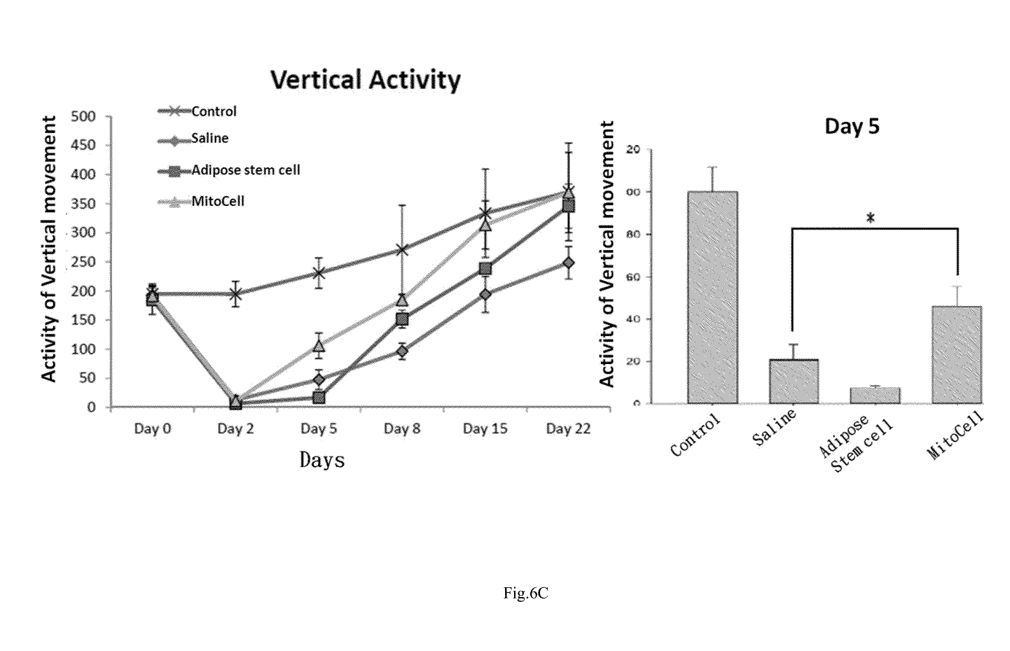

As shown in FIGS. 6A, 6B, and 6C, the balance and coordination abilities were decreased in mice after administration of MPTP (Group 2). However, after rejection of adipose stem cells or MitoCells (Groups 3 and 4), the number of vertical movement (FIG. 6A), time (FIG. 6B) and activities (FIG. 6C), preferably rejection of MitoCells (Group 4).

Mice were sacrificed with an excess dose of anesthetic (2-3 times the anesthetic dose). When mice were deeply anesthetized, mice were perfused with saline to wash out the blood and then with paraformaldehyde until all limbs became stiff to remove the brain of mice.

The skins behind ears were cut with the straight sharp scissor and the skin over the skull was vertically cut to expose the skull. The upper parts of the neck were cut by scissors to separate the neck bones and cerebellum and then the skulls were cut through the nose without cutting the brain, carefully. The parts below the brain were cleaned to remove the brain. The brain was dehydrated and placed on an operation table. The operation table was pre-cooled to prevent brain damages.

The cerebellum and olfactory bulb in brain were removed and the right and left sides of the brain were cut into two parts. The parts were embedded in optimal cutting temperature compound (OTC) and sectioned by a cryostat.

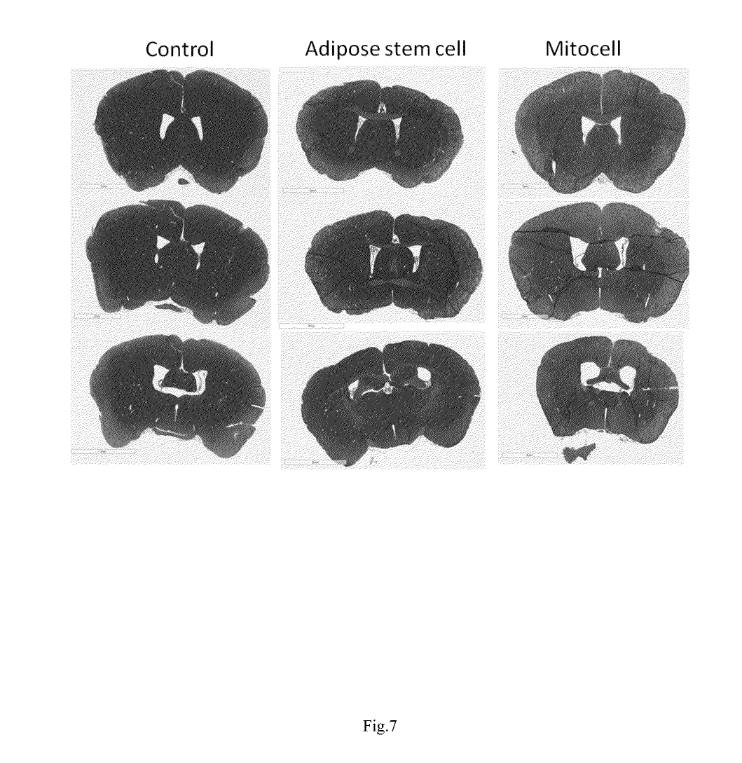

The brain sections stained by H&E were analyzed to determine the damages of inflammation response in brain cells. The results indicated that no damage or inflammation response was found in brain cells (FIG. 7).

1. A medium of culturing a stem cell, comprising an angelica extract.

2. The medium according to claim 1, wherein the angelica extract is butylidenephthalide.

3. The medium according to claim 2, wherein the angelica extract has a concentration of 5 to 160 μg/μl.

4. The medium according to claim 2, wherein the angelica extract has a concentration of 20 μg/μl.

5. A method for preparing a MitoCell, comprising culturing a cell in the medium of claim 1.

6. The method according to claim 1, wherein the stem cell is selected from a group consisting of adipose stem cells, neural stem cells, neural crest stem cells, mesenchymal stem cells, hematopoietic stem cells, pancreatic stem cells, skin stem cells, embryonic stem cells, endothelial stem cells, liver stem cells, intestinal epithelial stem cells and germ stem cells.

7. A MitoCell which is derived from a stem cell treated with the angelica extract, wherein the mitochondria of the MitoCell has a ratio of JC-1 staining red/green fluorescence of 6.5 to 2.7.

8. The MitoCell according to claim 7, wherein the stem cell is selected from a group consisting of adipose stem cells, neural stem cells, neural crest stem cells, mesenchymal stem cells, hematopoietic stem cells, pancreatic stem cells, skin stem cells, embryonic stem cells, endothelial stem cells, liver stem cells, intestinal epithelial stem cells and germ stem cells.

9. The MitoCell according to claim 7, wherein the mitochondria activity of the MitoCell is changed, and the MitoCell has high expression of cell marker Nuur1.

10. The MitoCell according to claim 7, wherein the MitoCell has a low express of cell marker IL8.

11. The MitoCell according to claim 7, wherein the MitoCell expresses a stem cell marker of CD44+ and CD105+.

12. A pharmaceutical composition for treating degenerative neurological disease, comprising the MitoCell of claim 7 and a pharmaceutically acceptable salt.

13. The pharmaceutical composition according to claim 11, wherein the MitoCell is present in an amount of 50 to 90% of the composition.

14. The pharmaceutical composition according to claim 11, wherein the MitoCell is present in an amount of 80 to 90% of the composition.

15. Use of MitoCell for preparing a pharmaceutical composition of treatment of degenerative neurological disease, wherein the MitoCell is a stem cell treated with an angelica extract.
